(19) United States
(12) Patent Application Publication
(10) Pub. No.: US 2026/0199378 A1
(43) Pub. Date: Jul. 16, 2026

(54) ORAL DELIVERY PRODUCT

(71) Applicant: KSHN POUCH CO., LLC, Berkley, MI (US)

(72) Inventor: Nir Saar, Berkley, MI (US)

(21) Appl. No.: 19/445,546

(22) Filed: Jan. 10, 2026

Related U.S. Application Data
(60) Provisional application No. 63/744,059, filed on Jan. 10, 2025.

(51) Int. Cl. A61K 31/00; A61K 9/00; A61K 9/107; A61K 47/02; A61K 47/12; A61K 47/26; A61K 47/44

(52) U.S. Cl. CPC A61K 31/658; A61K 9/009; A61K 9/1075; A61K 47/02; A61K 47/12; A61K 47/26; A61K 47/44

(57) ABSTRACT

An oral delivery product is disclosed. The oral delivery product comprises a delivery housing configured for placement in a mouth of a user and a composition disposed within the delivery housing. The composition comprises at least one phytocannabinoid, an oil, a surfactant, and a solid carrier or matrix, wherein the phytocannabinoid is dissolved or dispersed in the oil. The delivery housing permits contact between the composition and saliva when placed in the mouth, thereby delivering the phytocannabinoid to the user.

### CROSS-REFERENCE TO RELATED APPLICATIONS

This application claims priority to U.S. Provisional Application No. 63/744,059, filed on Jan. 10, 2025, which is incorporated herein by reference in its entirety and for all purposes.

### FIELD OF THE INVENTION

The present disclosure relates generally to an oral delivery product and, more specifically, to an oral delivery product including at least one phytocannabinoid.

### DESCRIPTION OF THE RELATED ART

Cannabis and marijuana products are known in the art and a focus of increasing development in view of social acceptance and changing laws. Historically, cannabis and marijuana products are smoked, which converts THCA present in raw cannabis to THC. However, smoking also introduces a number of undesirable and unwanted byproducts. As a result, many users desire alternative delivery mechanisms, including edibles or vaporizers. However, many of these alternative delivery mechanisms have their own drawbacks. By way of example, vaporizing requires a device for use, which may not always be available or otherwise charged for use. Additionally, many oral formats suffer from delayed onset, poor dispersibility, or undesirable taste, and it can be difficult to formulate phytocannabinoids in stable, user-acceptable oral products. Thus, there remains opportunity to provide alternative delivery options.

### BRIEF SUMMARY OF THE INVENTION

An oral delivery product is disclosed herein. The oral delivery product includes a delivery housing that is configured for placement in an oral cavity of a user. The oral delivery product further includes a composition disposed within the delivery housing. The composition comprises: (i) at least one phytocannabinoid; (ii) an oil; (iii) a solid carrier or matrix; and (iv) a surfactant. The at least one phytocannabinoid is dispersed in the oil. The oil and the surfactant are associated with the solid carrier or matrix to provide the composition. Upon contact with saliva, the composition releases at least a portion of the at least one phytocannabinoid for oral absorption. A method of preparing the oral delivery product is also disclosed.

### DETAILED DESCRIPTION OF THE INVENTION

An oral delivery product is disclosed herein. The oral delivery product includes (a) a delivery housing configured for placement in a mouth of a user and (b) a composition disposed within the delivery housing. The composition comprises: (i) at least one phytocannabinoid; (ii) an oil; (iii) a solid carrier or matrix; and (iv) a surfactant, wherein the phytocannabinoid is dispersed, optionally dissolved, in the oil in the composition. The delivery housing is capable of delivering the at least one phytocannabinoid to the user when the oral delivery product is at least partially present in the mouth of the user, e.g., sublingually. In certain embodiments, the composition may be considered a “dry” composition notwithstanding the presence of the oil and surfactant. For example, as described below, the solid carrier or matrix may have a significantly high surface area to volume ratio so as to allow impregnation or incorporation of the phytocannabinoid, oil, and surfactant in or on the solid carrier or matrix, including pores thereof.

The oral delivery product may be referred to as a pouch. The pouch is utilized by a user by placing the pouch in a mouth of the user, which allows for the release of at least some of the at least one phytocannabinoid and sublingual absorption thereof by the user. Typically, the at least one phytocannabinoid is released from the delivery housing in the presence of saliva or another liquid. The at least one phytocannabinoid need not be soluble in water; saliva can release the phytocannabinoid from the delivery housing even when the phytocannabinoid is insoluble in water or saliva.

Typically, the delivery housing comprises a fabric. The fabric may be woven or non-woven, and is not limited. Typically, the fabric is selected for compatibility with end use applications of the oral delivery product. In certain embodiments, the fabric does not degrade or otherwise dissolve in use, e.g. in the presence of saliva.

In specific embodiments, the fabric comprises, alternatively is, a cellulosic fabric, which may comprise individual cellulose fibers. The cellulosic fabric comprises, alternatively consists essentially of, alternatively consists of, a cellulose material. The cellulose material may comprise any matter derived from any plant source or may be synthetic. The cellulosic fabric may also include some water or moisture content, although the cellulosic fabric is typically dry, i.e., does not contain any free moisture content but for that which may be associated with the relative humidity in an environment in which the cellulosic fabric is prepared, derived, formed, and/or stored.

The cellulosic fabric typically comprises carbohydrate polymers (e.g. cellulose and/or hemicellulose), and may further comprise an aromatic polymer (e.g. lignin). The cellulosic fabric is typically a natural cellulosic material, i.e., is not synthetically derived. For example, the cellulosic fabric may be derived from wood (hardwood, softwood, and/or plywood).

Specific examples of suitable hardwoods from which the cellulosic fabric may be derived include, but are not limited to, ash, aspen, cottonwood, basswood, birch, beech, chestnut, gum, elm eucalyptus, maple, oak, poplar, sycamore, and combinations thereof. Specific examples of suitable softwoods from which the cellulosic fabric may be derived include, but are not limited to, spruce, fir, hemlock, tamarack, larch, pine, cypress, redwood, and combinations thereof. Combinations of different hardwoods, combinations of different softwoods, combinations of hardwood(s) and softwood(s), or combinations of one or both of hardwoods and softwoods with plywood, may be utilized together as the cellulosic fabric. The cellulosic fabric may be virgin, recycled, or a combination thereof.

The cellulosic fabric may have any form and size, e.g., from nanometer to millimeter diameters of individual fibers, which may be independently selected and randomized. The cellulosic fabric may be formed via a variety of techniques known to one of skill in the art, typically as a function of the form thereof.

Alternatively, the cellulosic fabric may comprise a polysaccharide, which may comprise both a cellulose or hemicellulose derivative and optionally lignin. Alternatively, the cellulosic fabric may consist essentially of, alternatively consist of polymers consisting of subunits of β-D glucose. Alternatively, the polysaccharide filler may comprise starches, pullulans, pectins, dextrans, alginates or animal derived polysaccharides such as chitin and chitosan.

In addition, the cellulosic fabric may comprise cellulosic fibers or material from other non-wood sources, such as cellulosic material from plants, or other plant-derived polymers, for example agricultural by-products, chaff, sisal, bagasse, wheat straw, kapok, ramie, henequen, corn fiber or coir, nut shells, flax, jute, hemp, kenaf, rice hulls, abaca, peanut hull, bamboo, straw, lignin, starch, or cellulose and cellulose-containing products, and combinations thereof. In certain embodiments, the delivery housing further comprises a starch.

The term “starch,” as used herein, may refer to pure starch from any source, modified starch, or starch derivatives. Starch is present, typically in granular form, in almost all green plants and in various types of plant tissues and organs (e.g., seeds, leaves, rhizomes, roots, tubers, shoots, fruits, grains, and stems). Starch can vary in composition, as well as in granular shape and size. Often, starch from different sources has different chemical and physical characteristics. A specific starch can be selected for inclusion in the composition based on the ability of the starch material to impart a specific organoleptic property to composition. Starches derived from various sources can be used. For example, major sources of starch include cereal grains (e.g., rice, wheat, and maize) and root vegetables (e.g., potatoes and cassava). Other examples of sources of starch include acorns, arrowroot, arracacha, bananas, barley, beans (e.g., favas, lentils, mung beans, peas, chickpeas), breadfruit, buckwheat, canna, chestnuts, colacasia, katakuri, kudzu, malanga, millet, oats, oca, Polynesian arrowroot, sago, sorghum, sweet potato, quinoa, rye, tapioca, taro, tobacco, water chestnuts, and yams. Certain starches are modified starches. A modified starch has undergone one or more structural modifications, often designed to alter its high heat properties. Some starches have been developed by genetic modifications, and are considered to be “genetically modified” starches. Other starches are obtained and subsequently modified by chemical, enzymatic, or physical means. For example, modified starches can be starches that have been subjected to chemical reactions, such as esterification, etherification, oxidation, depolymerization (thinning) by acid catalysis or oxidation in the presence of base, bleaching, transglycosylation and depolymerization (e.g., dextrinization in the presence of a catalyst), cross-linking, acetylation, hydroxypropylation, and/or partial hydrolysis. Enzymatic treatment includes subjecting native starches to enzyme isolates or concentrates, microbial enzymes, and/or enzymes native to plant materials, e.g., amylase present in corn kernels to modify corn starch. Other starches are modified by heat treatments, such as pregelatinization, dextrinization, and/or cold water swelling processes. Certain modified starches include monostarch phosphate, distarch glycerol, distarch phosphate esterified with sodium trimetaphosphate, phosphate distarch phosphate, acetylated distarch phosphate, starch acetate esterified with acetic anhydride, starch acetate esterified with vinyl acetate, acetylated distarch adipate, acetylated distarch glycerol, hydroxypropyl starch, hydroxypropyl distarch glycerol, and starch sodium octenyl succinate.

In other examples, the fabric comprises a polymer, which can be synthetic or natural. Non-limiting examples of suitable polymers include polyolefins (e.g. polyethylenes (PE), polypropylenes (PP), polybutylenes, poly(1-butene), polyisobutene, poly(l-pentene), poly(3-methyl-1-pentene), poly(4-methyl-1-hexene), poly(5-methyl-1-hexene), etc.), polycycloolefins, polyamides (e.g. nylon 6, nylon 66, etc.), polyesters, and combinations thereof. In specific embodiments, the polymer is selected from a polyethylene, a polypropylene, a polyamide, and a polyester. In one embodiment, the polymer is polyethylene. In another embodiments, the polymer is polypropylene. These polymers may alternatively be referred to as plastics. Additionally, elastomers and/or rubbers can be added to or compounded with the polymer to modify or improve properties.

Other examples of suitable polymers include polyvinyls (e.g. polyvinyl chloride, polyvinyl alcohol, polyvinyl ester (vinylester)), polycarbonates, polystyrenes, polyimides, polyphenylene sulfides; polyphenylene ethers; polyamideimides, polyoxymethylenes (POM), ethylene vinyl alcohol (EVOH), ethylene vinyl acetate (EVA), ethylene alkyl (meth)acrylates, ethylene propylene diene monomer (EPDM), ethylene propylene rubber (EPR), acrylics (e.g. polymethylmethacrylate), celluloses (e.g. triacetylcellulose, diacetylcellulose, cellophane, etc.), polyurethanes, polyetherimides (PEI); polysulfones (PSU); polyethersulfones; polyketones (PK); polyetherketones (PEK); polyetheretherketones (PEEK); polyetherketoneketones (PEKK); polyarylates (PAR); polyethernitriles (PEN); polysulfones; polyethersulfones; polyphenylsulfones; polyetherimides; resol-type; urea (e.g. melamine-type); ABS (acrylonitrile butadiene styrenic polymer), fluorinated resins, such as polytetrafluoroethylenes; thermoplastic vulcanizates; styrenics; thermoplastic elastomers, such as polystyrene types, polyolefin types, polyurethane types, polyester types, polyamide types, polybutadiene types, polyisoprene types, fluoro types, and the like; and copolymers, modifications, and combinations thereof.

The polymer may further comprise an elastomer. Non-limiting examples of elastomers include styrene-butadiene rubber, polyether urethane rubber, polyester urethane rubber, butyl rubber, nitrile rubber, chloroprene rubber (neoprene), polyacrylate rubber, ethylene acrylate rubber, ethylene-propylene rubber, ethylene-propylene-diene rubber, ethylene propylene diene monomer (EPDM), ethylene propylene rubber (EPR), fluorosilicone rubber, fluorocarbon rubber, perfluorianted elastomer, styrene butadiene rubber, chlorosulfonated polyethylene, polyisoprene rubber, polysulfide rubber, ethylene acrylate rubber, epichlorohydrine rubber, perfluoroelastomer (e.g. Kalrez™), polysulfide rubber, and combinations thereof.

Alternatively still, the fabric may comprise a silk mesh, or a blend, e.g. a cotton-polyester blend, or an alginate.

The oral delivery product further includes a composition disposed within the delivery housing comprising at least one phytocannabinoid that is dissolved or dispersed in an oil or emulsion with a surfactant and is associated with a solid carrier or matrix to form a composition. In such embodiments, the oil or emulsion may be adsorbed into the solid carrier or matrix. In other embodiments, the solid carrier or matrix comprises a porous inorganic material, such as a silica-based material, capable of retaining lipid-based systems.

The at least one phytocannabinoid is not particularly limited and may be any phytocannabinoid obtainable from plants of the genus Cannabis, including species of Cannabis sativa, Cannabis indica, and Cannabis ruderalis, as well as derivatives, variants, and combinations thereof. Other flowering plants may also be utilized, such as those from which one or more secondary metabolites may be extracted. As known in the art, when the phytocannabinoid comprises or is THC, the phytocannabinoid may have to be processed prior to use in the oral delivery product to ensure bioactivity. By way of example, THC is generally obtained from Cannabis plants via decarboxylation of THCA.

Specific examples of suitable phytocannabinoids include cannabidiol (CBD), cannabinodiol (CBDL), cannabinol propyl variant (CBNV), cannabitriol (CBO), tetrahydrocannabinol (THC), cannabinol (CBN), tetrahydrocannabinolic acid (THCA), tetrahydrocannabivarinic acid (THCV A), cannabidiolic acid (CBDA), cannabigerol (CBG), cannabichromene (CBC), cannabicyclol (CBL), cannabivarin (CBV), tetrahydrocannabivarin (THCV), cannabidivarin (CBDV), cannabichromevarin (CBCV), cannabigerovarin (CBGV), cannabigerol monomethyl ether (CBGM), cannabielsoin (CBE), cannabicitran (CBT), or combinations thereof. In certain embodiments, the phytocannabinoid comprises THC. The oral delivery product may include two or more different phytocannabinoids in combination with one another.

The phytocannabinoid may also or alternatively be hemp derived.

The phytocannabinoid may be present discretely or in a carrier or matrix. For example, in one embodiment, the phytocannabinoids may be utilized as an emulsion. Such emulsions are generally known in the art and include an oil. For example, the phytocannabinoid may be mixed with the oil and present in the emulsion as a discontinuous phase, with water or another liquid immiscible with the oil as the continuous phase. Alternatively, the oil may be the continuous phase.

Examples of the solid carrier or matrix include a material capable of immobilizing an oil or emulsion containing the phytocannabinoid. Suitable carriers or matrices include, but are not limited to, porous inorganic materials such as silica-based materials (e.g., amorphous silica, mesoporous silica, precipitated silica, fumed silica, hydrated silica, silica gel), silicate materials (e.g., calcium silicate, magnesium aluminum silicate, sodium aluminosilicate), carbohydrate-based materials (e.g., maltodextrin, modified starches, dextrins, gum arabic, cyclodextrins, cellulose), protein- or hydrocolloid-based materials, and combinations thereof, wherein the oil or emulsion is retained by adsorption, absorption, encapsulation, or physical entrapment. In specific embodiments, the solid carrier or matrix is pharmaceutical grade and suitable for ingestion.

In certain embodiments, the solid carrier or matrix comprises, alternatively is, a silica-based material. The silica-based material may have an average particle size of from greater than 0 to 500, alternatively from 5 to 250, alternatively from 25 to 200, μm. The average particle size is based on the largest dimension of each particle of silica-based material. In these or other embodiments, the silica-based material has a surface area of at least 100, alternatively at least 200, alternatively at least 250, m²/g. In these or other embodiments, the silica-based material has a pore volume of from greater than 0 to 5, alternatively from 0.1 to 2, alternatively from 0.25 to 1.75 mL/g.

In one embodiment, the oil may comprise a natural oil. Natural oils are oils that are not derived from petroleum. More specifically, natural oils are derived from animals and/or vegetative matter (including seeds and nuts). Common natural oils include triglycerides of mixtures of fatty acids, particularly mixtures containing some unsaturated fatty acids. In certain embodiments, the natural oil comprises a medium-chain triglyceride (MCT) oil. Such oils are suitable for dissolving or dispersing the phytocannabinoid and for forming a homogeneous liquid composition in combination with a surfactant prior to association with a solid carrier or matrix. Alternatively, the organic oil may be a derivative of a natural oil such as a transesterified vegetable oil, a boiled natural oil, a blown natural oil, or a stand oil (e.g. a thermally polymerized oil). The natural oil may be derived from a variety of sources and may comprise, for example, wheatgerm, sunflower, grapeseed, castor, shea, avocado, olive, soybean, sweet almond, palm, rapeseed, cotton seed, hazelnut, macadamia, jojoba, blackcurrant, evening primrose, and combinations thereof.

Alternatively to the liquids exemplified above, the organic oil may be a solid, such as a wax. When the organic oil comprises a wax, the wax typically has a melting point of from 30 to 100° C. The wax may be, for example, a hydrocarbon wax, such as a petroleum-derived wax; a wax comprising carboxylic esters, such as beeswax, lanolin, tallow, carnauba, candelilla, tribehenin; or a wax derived from plant seeds, fruits, nuts or kernel, including softer waxes referred to as ‘butter,’ such as mango butter, shea butter or cocoa butter. The wax may alternatively be a polyether wax or a silicone wax. Typically, however, the organic oil is a liquid.

The emulsion may include a surfactant, which generally interfaces between the continuous and discontinuous phase. The surfactant may alternatively be referred to as an emulsifier or a surface active agent. In certain embodiments, the surfactant is dispersed in an oil and contributes to the formation of a homogeneous liquid composition containing at least one phytocannabinoid.

The surfactant may be any surfactant capable of emulsifying the various components or improving stability of the emulsion. The surfactant may comprise a non-ionic surfactant, an anionic surfactant, a cationic surfactant, an amphoteric surfactant, or combinations thereof. The amount of the surfactant utilized may vary and is sufficient for forming the emulsion, as understood in the art.

In certain embodiments, the surfactant comprises a non-ionic surfactant. Non-limiting examples of suitable non-ionic surfactants include polyoxyethylene sorbitan fatty acid esters (e.g., polysorbate surfactants, such as polysorbate 80), ethoxylated castor oil derivatives (e.g., PEG-40 castor oil); condensates of ethylene oxide with an amine or an amide; condensation products of alkylene oxides (e.g. ethylene and propylene oxide); esters of glycerol; sucrose; sorbitol; fatty acid alkylol amides; sucrose esters; fluoro-surfactants; fatty amine oxides; polyoxyalkylene alkyl ethers, such as polyethylene glycol alkyl ether; polyoxyalkylene sorbitan ethers; polyoxyalkylene alkoxylate esters; polyoxyalkylene alkylphenol ethers; ethylene glycol propylene glycol copolymers and alkylpolysaccharides as described in U.S. Pat. No. 5,035,832, which is incorporated by reference in its entirety. Additional examples of non-ionic surfactants include polymeric surfactants, such as polyvinyl alcohol (PVA) and polyvinylmethylether. Non-ionic surfactants are commercially available from a variety of suppliers.

Examples of anionic surfactants include alkyl sulphates, such as lauryl sulphate; sulfonic acids; alkali metal sulforecinates; sulfonated glyceryl esters of fatty acids, such as sulfonated monoglycerides of coconut oil acids; salts of sulfonated monovalent alcohol esters; amides of amino sulfonic acids; sulfonated products of fatty acid nitriles; sulfonated aromatic hydrocarbons; condensation products of naphthalene sulfonic acids with formaldehyde; sodium octahydroanthracene sulfonate; alkali metal alkyl sulphates; ester sulphates; alkarylsulfonates; alkali metal soaps of higher fatty acids, alkylaryl sulphonates, such as sodium dodecyl benzene sulphonate; long chain fatty alcohol sulphates; olefin sulphates and olefin sulphonates; sulphated monoglycerides; sulphated esters; sulphonated ethoxylated alcohols; sulphosuccinates; alkane sulphonates; phosphate esters; alkyl isethionates; alkyl taurates; and alkyl sarcosinates.

Further examples of nonionic surfactants include polyoxyethylene alkyl ethers, polyoxyethylene alkylphenol ethers, polyoxyethylene lauryl ethers, polyoxyethylene sorbitan monoleates, polyoxyethylene alkyl esters, polyoxyethylene sorbitan alkyl esters, polyethylene glycol, polypropylene glycol, diethylene glycol, ethoxylated trimethylnonanols, polyoxyalkylene-substituted silicones (rake or ABn types), silicone alkanolamides, silicone esters, silicone glycosides, and mixtures thereof.

Nonionic surfactants include dimethicone copolyols, fatty acid esters of polyols, for instance sorbitol or glyceryl mono-, di-, tri- or sesqui-oleates or stearates, glyceryl or polyethylene glycol laurates; fatty acid esters of polyethylene glycol (polyethylene glycol monostearate or monolaurate); polyoxyethylenated fatty acid esters (stearate or oleate) of sorbitol; polyoxyethylenated alkyl (lauryl, cetyl, stearyl or octyl) ethers.

Anionic surfactants include carboxylates (sodium 2-(2-hydroxyalkyloxy)acetate), amino acid derivatives (N-acylglutamates, N-acylgly-cinates or acylsarcosinates), alkyl sulfates, alkyl ether sulfates and oxyethylenated derivatives thereof, sulfonates, isethionates and N-acylisethionates, taurates and N-acyl N-methyltaurates, sulfosuccinates, alkylsulfoacetates, phosphates and alkyl phosphates, polypeptides, anionic derivatives of alkyl polyglycoside (acyl-D-galactoside uronate), and fatty acid soaps, and mixtures thereof.

Amphoteric and zwitterionic surfactants include betaines, N-alkylamidobetaines and derivatives thereof, proteins and derivatives thereof, glycine derivatives, sultaines, alkyl polyaminocarboxylates and alkylamphoacetates, and mixtures thereof.

In certain embodiments, the composition is in the form of a powder. As used herein, the term “powder” refers to a solid composition comprising particulate material suitable for handling, storage, and incorporation into the delivery housing. In certain embodiments, the powder is free-flowing and capable of being distributed uniformly within the delivery housing. In other embodiments, the powder comprises particles formed by association of a phytocannabinoid-containing oil and surfactant with a solid carrier or matrix, thereby providing a dry, physical form. In certain embodiments, the powder is substantially free of agglomerates.

In some embodiments, the phytocannabinoid is present in the oral delivery product in an amount of at least about 0.001% by weight based on the total weight of the composition, such as in a range from about 0.001% to about 20% based on the total weight of the composition. In other embodiments, the phytocannabinoid is present in the oral product in a concentration of from about 0.1% to about 15% by weight, based on the total weight of the composition. In yet other embodiments, the phytocannabinoid is present in a concentration from about 1% to about 15% by weight, such as from about 5% to about 15% by weight, based on the total weight of the composition. In further other embodiments, the phytocannabinoid is present in the oral product in a concentration of from about 0.5% to about 10% by weight, such as from about 1% to about 7.5% by weight, such as from 1.5% to about 5% by weight, such as from about 1.5% to about 2.5% by weight, based on the total weight of the composition.

In some embodiments, the weight ratio of the delivery housing to the phytocannabinoid is from about 5:1 to about 100:1, such as from about 10:1 to about 60:1, such as from about 15:1 to about 50:1, such as from about 20:1 to about 40:1, such as from about 25:1 to about 35:1. In some embodiments, the weight ratio of microcrystalline cellulose to cannabidiol is from about 5:1 to about 100:1, such as from about 10:1 to about 60:1, such as from about 15:1 to about 50:1, such as from about 20:1 to about 40:1, such as from about 25:1 to about 35:1.

In certain embodiments, the oil and the surfactant are present in the composition in a mass ratio of from about 1:1 to about 3:1, surfactant to oil. In other embodiments, the oil and the surfactant are present in a mass ratio of from about 1.5:1 to about 2.5:1, surfactant to oil. In yet other embodiments, the oil and the surfactant are present in a mass ratio of about 2:1, surfactant to oil.

In some embodiments, the composition comprises from about 30% to about 60% by weight solid carrier or matrix, based on the total mass of the phytocannabinoid, the oil, the surfactant, and the solid carrier or matrix. In other embodiments, the composition comprises from about 35% to about 55% by weight solid carrier or matrix, based on the total mass of the phytocannabinoid, the oil, the surfactant, and the solid carrier or matrix. In yet other embodiments, the solid carrier or matrix is present in an amount sufficient to provide a dry solid physical form to the composition.

In certain embodiments, the composition comprises from about 10% to about 50% by weight surfactant, based on the total mass of the phytocannabinoid, the oil, the surfactant, and the solid carrier or matrix. In certain embodiments, the surfactant comprises PEG-40 castor oil, polysorbate 80, or combinations thereof, and is present in an amount from about 15% to about 40% by weight based on the total mass of phytocannabinoid, the oil, the surfactant, and the solid carrier or matrix.

In some embodiments, the composition comprises from about 5% to about 20% by weight oil, based on the total mass of the phytocannabinoid, the oil, the surfactant, and the solid carrier or matrix. In certain embodiments, the oil comprises medium-chain triglycerides (MCT) oil and is present in an amount from about 7% to about 15% by weight based on the total mass of phytocannabinoid, the oil, the surfactant, and the solid carrier or matrix.

In certain embodiments, the oral delivery product further includes at least one optional additive. The optional additive may be an active ingredient or inactive ingredient. Examples of the optional additive includes, for example, fibers/filler (e.g. hemp fiber, PLA fiber, which may be derived from corn, banana fiber, etc.), flavors, minerals, amino acids, stimulants, botanical ingredients (e.g., hemp, lavender, peppermint, eucalyptus, rooibos, fennel, cloves, chamomile, basil, rosemary, clove, citrus, ginger, cannabis, ginseng, maca, and tisanes), stimulants (e.g., caffeine or guarana), amino acids (e.g., taurine, theanine, phenylalanine, tyrosine, and tryptophan), vitamins (e.g. B6, B12, and C), antioxidants, nicotine components, pharmaceutical ingredients (e.g., nutraceutical and medicinal ingredients), melatonin, mushrooms or mushroom extracts, sweeteners (e.g. sorbitol, xylitol, sugar) and high-intensity sweeteners (e.g. aspartame, ace k, sucralose, monk fruit), flavor masking agents, pH adjusters or other supplements or additives.

In certain embodiments, one or more optional additives are provided as a separate solid composition. In such embodiments, the separate solid composition comprising the optional additives is combined with the composition comprising the phytocannabinoid, the oil, the surfactant, and the solid carrier or matrix. The resulting combined composition remains a dry, solid physical form suitable for disposition in or on the delivery housing.

In some embodiments, the optional additive comprises a filler. The filler may be untreated, pretreated, or added in conjunction with an optional filler treating agent, which when so added may treat the filler in situ or prior to incorporation of the filler in the oral delivery product. The filler may be a single filler or a combination of two or more fillers that differ in at least one property such as type of filler, method of preparation, treatment or surface chemistry, filler composition, filler shape, filler surface area, average particle size, and/or particle size distribution.

The shape and dimensions of the filler is also not specifically restricted. For example, the filler may be spherical, rectangular, ovoid, irregular, and may be in the form of, for example, a powder, a flour, a fiber, a flake, a chip, a shaving, a strand, a scrim, a wafer, a wool, a straw, a particle, and combinations thereof. Dimensions and shape are typically selected based on the type of the filler utilized, and the selection of other components utilized.

Non-limiting examples fillers include silica (e.g. fumed, ground, precipitated), hydrated dolomite, silicone resin, wollastonite, soapstone, kaolinite, kaolin, mica muscovite, phlogopite, halloysite (hydrated alumina silicate), aluminum silicate, sodium aluminosilicate, glass (fiber, beads or particles, including recycled glass, e.g. from wind turbines or other sources), clay, magnetite, hematite, calcium carbonate such as precipitated, fumed, and/or ground calcium carbonate, zinc oxide, talc, diatomaceous earth, clays, mica, chalk, titanium dioxide (titania), zirconia, coal, lignite, charcoal, activated carbon, non-functional silicone resin, and combinations thereof.

Specific examples of flavors, which may be botanical ingredients or otherwise sourced, include mint, sweetmint, berry cherry, citrus, spearmint, peppermint, wintergreen, cinnamon, bubblegum, watermelon, strawberry, cherry, grape, raspberry, lemon, orange, pineapple, hemp, eucalyptus, rooibos, fennel, cloves, chamomile, basil, rosemary, coconut, clove, ginseng, tisanes, apple, mango, blueberry, tropical punch, pomegranate, peach, lime, ginger, honey, green tea, cotton candy, fruit punch, banana, coconut, cola, root beer, lychee, melon, kiwi, passionfruit, pink lemonade, dragon fruit, chocolate, coffee, guava, caramel, vanilla, chili, mint chocolate chip, blood orange, lemonade, pear, chai, matcha, cranberry, pumpkin spice, tangerine, mocha, cucumber, lavender, ginger, butterscotch, and combinations thereof.

Examples of vitamins include a variety of different organic compounds such as alcohols, acids, sterols, and quinones. They may be classified into two solubility groups: lipid-soluble vitamins and water-soluble vitamins. Lipid-soluble vitamins that have utility in personal care formulations include retinol (vitamin A), ergocalciferol (vitamin D2), cholecalciferol (vitamin D3), phytonadione (vitamin K1), and tocopherol (vitamin E). Water-soluble vitamins that have utility in personal care formulations include ascorbic acid (vitamin C), thiamin (vitamin B1) niacin (nicotinic acid), niacinamide (vitamin B3), riboflavin (vitamin B2), pantothenic acid (vitamin B5), biotin, folic acid, pyridoxine (vitamin B6), and cyanocobalamin (vitamin B12). Additional examples of vitamins include derivatives of vitamins such as retinyl palmitate (vitamin A palmitate), retinyl acetate (vitamin A acetate), retinyl linoleate (vitamin A linoleate), and retinyl propionate (vitamin A propionate), tocopheryl acetate (vitamin E acetate), tocopheryl linoleate (vitamin E linoleate), tocopheryl succinate (vitamin E succinate), tocophereth-5, tocophereth-10, tocophereth-12, tocophereth-18, tocophereth-50 (ethoxylated vitamin E derivatives), PPG-2 tocophereth-5, PPG-5 tocophereth-2, PPG-10 tocophereth-30, PPG-20 tocophereth-50, PPG-30 tocophereth-70, PPG-70 tocophereth-100 (propoxylated and ethoxylated vitamin E derivatives), sodium tocopheryl phosphate, ascorbyl palmitate, ascorbyl dipalmitate, ascorbyl glucoside, ascorbyl tetraisopalmitate, tetrahexadecyl ascorbate, ascorbyl tocopheryl maleate, potassium ascorbyl tocopheryl phosphate, tocopheryl nicotinate, and mixtures thereof.

Specific examples of vitamins include vitamin A (e.g. retinol), vitamin B1 (thiamine), vitamin B2 (riboflavin), vitamin B3 (niacin, niacinamide), vitamin B5 (pantothenic acid, pantethine), vitamin B6 (pyridoxine, pyridoxal-5-phosphate (P5P)), vitamin B7 (biotin or D-bioton), vitamin B9 (folate/folic acid), vitamin B12 (cobalamin, methylcobalamin), vitamin C (ascorbic acid, ascorbate, L-ascorbic acid), vitamin D (e.g. calcitriol), vitamin E (e.g. alpha-tocopherol), vitamin K, choline, inositol, vitamin B complex, beta-carotene, vitamin D2 (e.g. ergocalciferol), vitamin D3 (e.g. cholecalciferol), K2 (e.g. menaquinone), zinc, and/or magnesium.

Specific examples of mushrooms for purposes of mushroom extracts or powders as an optional additive include reishi, lion's mane, chaga, cordyceps, turkey tail, maitake, shiitake, agaricus blazei, tremella, and/or poria. These mushrooms serve different purposes and can be selectively chosen for inclusion based on desired effects from use of the oral delivery product.

Masking agents include a variety of different compounds capable of modifying, reducing, or altering the perception of undesirable organoleptic properties associated with the phytocannabinoid. Masking agents may be classified into functional groups including cooling agents, sweeteners, acidulants and buffers, flavorants, bitterness blockers, and encapsulation or complexing agents. Cooling agents suitable for use herein include menthol, menthol crystals, peppermint oil, spearmint oil, eucalyptus oil, wintergreen oil, and synthetic cooling agents such as WS-3, WS-5, WS-23, WS-30, and combinations thereof. Sweeteners suitable for use as masking agents include monk fruit, reb a, reb m, tapioca syrup solids, xylitol, sorbitol, mannitol, erythritol, stevioside, sucralose, aspartame, acesulfame potassium, saccharin and salts thereof, and combinations thereof. Acidulants and buffers suitable for masking include citric acid, malic acid, tartaric acid, lactic acid, fumaric acid, citrates, sodium bicarbonate, potassium carbonate, and combinations thereof. Flavorants suitable for masking include mint, citrus, fruit, spice, herbal, and botanical flavors and extracts, whether natural or synthetic, as well as encapsulated flavor systems. Additional masking agents include bitterness blockers, cyclodextrins, maltodextrin-based systems, gum arabic, modified starches, polymeric encapsulation systems, and the like.

Specific examples of masking agents include menthol, menthol crystals, peppermint, spearmint, eucalyptus, wintergreen, mint blends, citrus flavor systems, fruit flavor systems, cooling agents such as WS-3, WS-5, WS-23, WS-30 sweeteners such as xylitol and stevioside, acids such as citric, and malic acid, buffers such as sodium citrate and sodium bicarbonate, cyclodextrin complexes, encapsulated flavor particles, and the like.

Specific examples of the pH adjuster include lactic acid, citric acid, glycolic acid, succinic acid, tartaric acid, dl-malic acid, potassium carbonate, sodium bicarbonate, sodium citrate, ammonium hydrogen carbonate, and the like.

Specific examples of sweeteners include sucralose, aspartame, salts of acesulfame, such as acesulfame potassium, alitame, saccharin and its salts, cyclamic acid and its salts, glycyrrhizin, dihydrochalcones, thaumatin, monellin, stevioside, xylitol and the like.

Examples of other optional additives include ashwagandha, Rhodiola rosea, turmeric (curcumin), elderberry, ginseng, Ginkgo biloba, spirulina, chlorella, green tea extract, GABA, L-theanine, omega-3 fish oil, resveratrol, milk thistle, coenzyme Q10 (CoQ10), saw palmetto, collagen, aloe vera, hyaluronic acid, astaxanthin, evening primrose oil, glucosamine, melatonin, probiotics, kava kava, berberine, L-arginine, bee pollen, quercetin, and/or amino acids (including BCAAs).

Examples of vegetable or botanical extracts are derived from plants (herbs, roots, flowers, fruits, or seeds) in oil or water soluble form, such as coconut, green tea, white tea, black tea, horsetail, Ginkgo biloba, sunflower, wheat germ, seaweed, olive, grape, pomegranate, aloe, apricot kernel, apricot, carrot, tomato, tobacco, bean, potato, adzuki bean, catechu, orange, cucumber, avocado, watermelon, banana, lemon or palm. Examples of herbal extracts include dill, horseradish, oats, neem, beet, broccoli, tea, pumpkin, soybean, barley, walnut, flax, ginseng, poppy, avocado, pea, sesame, and mixtures thereof.

Other examples of optional additives or ingredients include any of those disclosed in U.S. Publ. Pat. Appln. No. 2021/0307375, which is incorporated by reference herein in its entirety.

Other examples of optional additives include antibacterial agents, antifungal agents, therapeutic active agents, external analgesics, diuretics, agents for treating gastric and duodenal ulcers, proteolytic enzymes, antihistamine or H1 histamine blockers, sedatives, bronchodilators, diluents, and others. Additional components that may be used include antibiotics, antiseptics, antibacterial agents, anti-inflammatory agents, astringents, hormones, smoking cessation compositions, cardiovascular agents, antiarrhythmic agents, alpha-I blockers, beta blockers, ACE inhibitors, antiaggregants, antioxidants, non-steroidal anti-inflammatory agents such as diclofenac, antipsoriasis agents such as clobetasol propionate, antidermatitis agents, tranquilizer, anticonvulsants, anticoagulant agents, healing factors, proteins, cell growth nutrients, peptides, corticosteroidal drugs, antipruritic agents and others.

Examples of proteins or amino-acids and their derivatives include those extracted from wheat, soy, rice, corn, keratin, elastin or silk. Proteins may be in the hydrolyzed form and they may also be quaternized, such as hydrolyzed elastin, hydrolyzed wheat powder, hydrolyzed silk. Examples of protein include enzymes such as hydrolases, cutinases, oxidases, transferases, reductases, hemicellulases, esterases, isomerases, pectinases, lactases, peroxidases, laccases, catalases, and mixtures thereof. Examples of hydrolases include proteases (bacterial, fungal, acid, neutral or alkaline), amylases (alpha or beta), lipases, mannanases, cellulases, collagenases, lisozymes, superoxide dismutase, catalase, and mixtures thereof.

Examples of antioxidants are acetyl cysteine, arbutin, ascorbic acid, ascorbic acid polypeptide, ascorbyl dipalmitate, ascorbyl methylsilanol pectinate, ascorbyl palmitate, ascorbyl stearate, BHA, p-hydroxyanisole, BHT, t-butyl hydroquinone, caffeic acid, Camellia sinensis oil, chitosan ascorbate, chitosan glycolate, chitosan salicylate, chlorogenic acids, cysteine, cysteine HCl, decyl mercaptomethylimidazole, erythorbic acid, diamylhydroquinone, di-t-butylhydroquinone, dicetyl thiodipropionate, dicyclopentadiene/t-butylcresol copolymer, digalloyl trioleate, dilauryl thiodipropionate, dimyristyl thiodipropionate, dioleyl tocopheryl methylsilanol, isoquercitrin, diosmine, disodium ascorbyl sulfate, disodium rutinyl disulfate, distearyl thiodipropionate, ditridecyl thiodipropionate, dodecyl gallate, ethyl ferulate, ferulic acid, hydroquinone, hydroxylamine HCl, hydroxylamine sulfate, isooctyl thioglycolate, kojic acid, madecassicoside, magnesium ascorbate, magnesium ascorbyl phosphate, melatonin, methoxy-PEG-7 rutinyl succinate, methylene di-t-butylcresol, methylsilanol ascorbate, nordihydroguaiaretic acid, octyl gallate, phenylthioglycolic acid, phloroglucinol, potassium ascorbyl tocopheryl phosphate, thiodiglycolamide, potassium sulfite, propyl gallate, rosmarinic acid, rutin, sodium ascorbate, sodium ascorbyl/cholesteryl phosphate, sodium bisulfite, sodium erythorbate, sodium metabisulfide, sodium sulfite, sodium thioglycolate, sorbityl furfural, tea tree (Melaleuca aftemifolia) oil, tocopheryl acetate, tetrahexyldecyl ascorbate, tetrahydrodiferuloylmethane, tocopheryl linoleate/oleate, thiodiglycol, tocopheryl succinate, thiodiglycolic acid, thioglycolic acid, thiolactic acid, thiosalicylic acid, thiotaurine, retinol, tocophereth-5, tocophereth-10, tocophereth-12, tocophereth-18, tocophereth-50, tocopherol, tocophersolan, tocopheryl linoleate, tocopheryl nicotinate, tocoquinone, o-tolyl biguanide, tris(nonylphenyl) phosphite, ubiquinone, zinc dibutyldithiocarbamate, and mixtures thereof.

The particular content of optional additives present will vary depending upon the desired characteristics of the particular oral delivery product. Typically, an optional additive or combination thereof is present in a total concentration of at least about 0.001% by weight based on the total weight of the oral delivery product, such as in a range from about 0.001% to about 20%. In some embodiments, the optional additive is present in a concentration from about 0.1% w/w to about 10% by weight, such as, e.g., from about 0.5% w/w to about 10%, from about 1% to about 10%, from about 1% to about 5% by weight, based on the total weight of the oral delivery product. In some embodiments, the active ingredient or combination of active ingredients is present in a concentration of from about 0.001%, about 0.01%, about 0.1%, or about 1%, up to about 20% by weight, such as, e.g., from about 0.001%, about 0.002%, about 0.003%, about 0.004%, about 0.005%, about 0.006%, about 0.007%, about 0.008%, about 0.009%, about 0.01%, about 0.02%, about 0.03%, about 0.04%, about 0.05%, about 0.06%, about 0.07%, about 0.08%, about 0.09%, about 0.1%, about 0.2%, about 0.3%, about 0.4%, about 0.5% about 0.6%, about 0.7%, about 0.8%, or about 0.9%, to about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, or about 20% by weight, based on the total weight of the oral delivery product.

In some embodiments, the oral delivery product comprises one or more sweeteners. In such embodiments, the one or more sweeteners are present in an amount of from about 10% to about 80% by weight based on the total weight of the composition. In certain embodiments, the one or more sweeteners comprise a sugar alcohol and are present in an amount of from about 30% to about 85% by weight. In further embodiments, the one or more sweeteners comprise xylitol, which is present in an amount of from about 40% to about 80% by weight, based on the total weight of the oral delivery product. In still further embodiments, the sweetener further comprises a sweetener selected from stevioside and combinations thereof, present in an amount of from about 0.1% to about 5% by weight.

In certain embodiments, the oral delivery product further comprises one or more fillers or processing aids. In such embodiments, the one or more fillers or processing aids are present in an amount of from about 0.1% to about 10% by weight based on the total weight of the composition. In certain embodiments, the filler or processing aid comprises magnesium stearate and is present in an amount of from about 0.1% to about 5% by weight.

In certain embodiments, the oral delivery product comprises one or more flavorants. In such embodiments, the one or more flavorants are present in an amount of from about 1% to about 25% by weight based on the total weight of the composition. In certain embodiments, the one or more flavorants are selected from mint flavors, sweetmint flavors, spearmint flavors, citrus flavors, tropical flavors, coconut flavors, and combinations thereof, and are present in an amount of from about 5% to about 20% by weight.

In some embodiments, the oral delivery product comprises one or more masking agents. In such embodiments, the one or more masking agents are present in an amount of from about 0.01% to about 10% based on the total weight of the composition. In certain embodiments, the masking agent comprises menthol or menthol crystals and is present in an amount of from about 0.01% to about 1% by weight. In other embodiments, the masking agent comprises a flavor masking system and is present in an amount of from about 0.1% to about 5% by weight.

In certain embodiments, the oral delivery product comprises one or more acids, buffers, or pH adjusters. In such embodiments, the one or more acids, buffers, or pH adjusters are present in an amount of from about 0.05% to about 10% based on the total weight of the composition. In certain embodiments, the one or more acids, buffers, or pH adjusters are selected from citric acid, malic acid, sodium citrate, sodium bicarbonate, and combinations thereof, and are present in an amount of from about 0.1% to about 6% by weight.

Although there are no particular limitations on the method of preparing the oral delivery product described herein, methods including the following steps may be employed. Such methods are provided as non-limiting examples and may be modified as appropriate depending on the desired formulation, scale, or processing equipment.

In certain embodiments, a method of preparing the oral delivery product comprises a first step of combining at least one phytocannabinoid, an oil, and a surfactant to form a liquid composition. In such embodiments, the phytocannabinoid may be dissolved or dispersed in the oil prior to or during combination with the surfactant. The oil and surfactant may be mixed until a substantially homogeneous liquid composition is obtained.

In a second step, the liquid composition formed in the first step is combined with a solid carrier or matrix. In certain embodiments, the liquid composition is added to the solid carrier or matrix while mixing to promote distribution of the liquid composition throughout the solid carrier or matrix. Mixing may be continued until a dry, free-flowing solid composition is obtained. Freeze-drying may be utilized in addition to mixing so as to help produce such a free-flowing solid composition or powder.

In certain embodiments, the composition obtained in the second step, comprising the phytocannabinoid, the oil, the surfactant, and the solid carrier or matrix, is combined with one or more optional additive compositions. In such embodiments, the optional additives are provided as one or more separate solid or powder compositions, which may include sweeteners, flavorants, masking agents, acids, buffers, or other components described herein. The composition and the one or more optional additive compositions are combined by mixing to form a uniform, dry, solid physical form suitable for disposition in or on the delivery housing. In certain embodiments, the optional additive compositions are prepared as one or more pre-blends and then combined with the silica-adsorbed SNEDDS powder.

In certain embodiments, the combined composition is introduced into the delivery housing. In such embodiments, the delivery housing comprises a pouch or other enclosure suitable for oral placement, and the composition is disposed within the delivery housing prior to use. The composition may be introduced into the delivery housing by any suitable filling or loading technique, and the delivery housing may thereafter be sealed or otherwise closed to retain the composition while permitting contact with saliva during use.

In certain embodiments, the resulting composition retains the ability to form a nano emulsion upon contact with saliva or another aqueous medium when placed in the oral cavity of a user.

The above description relates to general and specific embodiments of the disclosure. However, various alterations and changes can be made without departing from the spirit and broader aspects of the disclosure as defined in the appended claims, which are to be interpreted in accordance with the principles of patent law including the doctrine of equivalents. As such, this disclosure is presented for illustrative purposes and should not be interpreted as an exhaustive description of all embodiments of the disclosure or to limit the scope of the claims to the specific elements illustrated or described in connection with these embodiments. Any reference to elements in the singular, for example, using the articles “a,” “an,” “the,” or “said,” is not to be construed as limiting the element to the singular.

Likewise, it is also to be understood that the appended claims are not limited to express and particular compounds, compositions, or methods described in the detailed description, which may vary between particular embodiments that fall within the scope of the appended claims. With respect to any Markush groups relied upon herein for describing particular features or aspects of various embodiments, different, special, and/or unexpected results may be obtained from each member of the respective Markush group independent from all other Markush members. Each member of a Markush group may be relied upon individually and or in combination and provides adequate support for specific embodiments within the scope of the appended claims.

Further, any ranges and subranges relied upon in describing various embodiments of the present invention independently and collectively fall within the scope of the appended claims, and are understood to describe and contemplate all ranges including whole and/or fractional values therein, even if such values are not expressly written herein. One of skill in the art readily recognizes that the enumerated ranges and subranges sufficiently describe and enable various embodiments of the present invention, and such ranges and subranges may be further delineated into relevant halves, thirds, quarters, fifths, and so on. As just one example, a range “of from 0.1 to 0.9” may be further delineated into a lower third, i.e., from 0.1 to 0.3, a middle third, i.e., from 0.4 to 0.6, and an upper third, i.e., from 0.7 to 0.9, which individually and collectively are within the scope of the appended claims, and may be relied upon individually and/or collectively and provide adequate support for specific embodiments within the scope of the appended claims. In addition, with respect to the language which defines or modifies a range, such as “at least,” “greater than,” “less than,” “no more than,” and the like, it is to be understood that such language includes subranges and/or an upper or lower limit. As another example, a range of “at least 10” inherently includes a subrange of from at least 10 to 35, a subrange of from at least 10 to 25, a subrange of from 25 to 35, and so on, and each subrange may be relied upon individually and/or collectively and provides adequate support for specific embodiments within the scope of the appended claims. Finally, an individual number within a disclosed range may be relied upon and provides adequate support for specific embodiments within the scope of the appended claims. For example, a range “of from 1 to 9” includes various individual integers, such as 3, as well as individual numbers including a decimal point (or fraction), such as 4.1, which may be relied upon and provide adequate support for specific embodiments within the scope of the appended claims.

The invention has been described in an illustrative manner, and it is to be understood that the terminology which has been used is intended to be in the nature of words of description rather than of limitation. Obviously, many modifications and variations of the present invention are possible in light of the above teachings. The invention may be practiced otherwise than as specifically described.

The following examples are illustrative of the present invention and should not be considered as limiting the scope of the invention.

The following Examples were prepared using commercially available materials unless otherwise indicated in the examples described herein.

In the Examples described herein, the silica-based carrier or matrix comprises a mesoporous, amorphous silica material commercially available under the trademark SYLOID® XDP. Equivalent silica-based carriers may be substituted.

As used in Table 1, “THC Oil” refers to a phytocannabinoid distillate comprising THC.
Examples 1-3 illustrate various materials and methods for preparing intermediate ingredients.
Example 4 discloses the preparation of a dry Self-Nanoemulsifying Phytocannabinoid Composition prepared according the procedure outlined in Examples 1-2.
Example 5 discloses a number of different oral delivery products and their respective compositions prepared according to Example 3.

### Example 1: Preparation of a Self-Nanoemulsifying Phytocannabinoid Composition

A self-nanoemulsifying drug delivery system composition (herein “SNEDDS”) is prepared as an anhydrous, homogeneous mixture of oils and surfactants. A phytocannabinoid distillate comprising tetrahydrocannabinol (“THC”) is heated to approximately 72° C. for two or more hours to reduce viscosity and facilitate handling. A surfactant comprising PEG-40 castor oil or polysorbate 80 and a lipid carrier comprising medium-chain triglyceride (“MCT”) oil are separately provided. The MCT oil is combined with the PEG-40 castor oil or polysorbate 80 and heated to approximately 25° C. while stirring for at least 5 minutes to form a uniform oil phase. The heated THC distillate is combined with the oil phase, and cannabidiol (“CBD”) is added. The resulting mixture is stirred and heated on a hot plate set to approximately 25° C., while maintaining a minimum temperature of 20° C., for at least 5 minutes, until a visually homogeneous composition is obtained. The resulting composition forms an oil-in-water nano emulsion upon dilution in an aqueous medium.

### Example 2: Adsorption of SNEDDS Composition into a Silica Carrier Matrix

The SNEDDS composition of Example 1 is combined with a porous inorganic carrier comprising a silica-based material. The liquid SNEDDS is poured into a mixing vessel containing the silica carrier and manually mixed to ensure initial wetting of the powder. The mixture is further mixed until the liquid composition is fully adsorbed into the carrier, forming a dry solid composition capable of forming a nanoemulsion upon exposure to saliva or another aqueous medium. The resulting powder is passed through an 18-mesh to 24-mesh screen to remove agglomerates and allowed to cool to room temperature. The solid SNEDDS composition retains nanoemulsifying properties upon exposure to saliva or another aqueous medium.

### Example 3: Preparation of a Finished Oral Delivery Composition Including Masking Agents

A sweetener pre-blend is prepared by combining xylitol, stevioside, and magnesium stearate, followed by grinding for approximately 1 minute to obtain a uniform powder. Separately, a flavor pre-blend is prepared by combining one or more flavoring agents, flavor masking agents, acids, and functional additives. The flavor pre-blend includes citric acid, malic acid, sodium citrate, sodium bicarbonate, vitamin C, chitosan lactate, menthol, and optional vitamins or minerals. Menthol is provided in the form of menthol crystals, which are pre-ground into a fine powder prior to incorporation.

The silica-adsorbed SNEDDS composition of Example 2 and the sweetener pre-blend are added to the flavor pre-blend and mixed. The combined composition is mixed using a paddle mixer for approximately 5 minutes with mixing paused partway through to ensure uniform distribution. The resulting composition is visually inspected to confirm homogeneity and absence of clumps. If needed, the composition is sieved through an 18-mesh screen.

Representative oral delivery compositions were prepared according to the procedures described above. The compositions are shown in Tables 2-5.

### Example 4: Preparation of a Self-Nanoemulsifying Drug Delivery System (SNEDDS)

A representative SNEDDS composition was prepared according to the procedure of Examples 1 and had the composition shown in Table 1. The SNEDDS composition was subsequently adsorbed into a silica-based carrier to form the solid composition according to the procedure of Example 2 and shown in Table 1.

TABLE 1
Representative SNEDDS Composition

| | Content in weight |
|---|---|
| Component | percent |
| Polysorbate 80 Oil | 29.4 |
| MCT Oil | 14.80 |
| CBD | 0.30 |
| THC Oil | 15.5 |
| Silica-Based Carrier | 40.00 |

### Example 5-Representative Finished Oral Delivery Compositions

Each of the representative finished oral delivery compositions shown in Tables 2-5 was prepared according to the procedures of Example 3 by combining the SNEDDS composition depicted in Table 1 with one or more separate solid compositions comprising sweeteners, flavorants, masking agents, acids, buffers, and optional additives as listed in Tables 2-5. In Tables 2-5, the basis for the weight percents is the overall combination of those listed components for each Representative Oral Delivery Composition.

In such embodiments, the SNEDDS composition was first associated with a silica-based carrier to form a composition, and the resulting composition was thereafter combined with the one or more separate solid compositions to form the finished oral delivery composition.

TABLE 2
Representative Oral Delivery Composition A

| | Content in weight |
|---|---|
| Component | percent |
| Xylitol | 54.96 |
| Magnesium Stearate | 1.00 |
| Stevioside | 1.50 |
| SNEDDS Composition | 13.53 |
| Tropical | 16.00 |
| Flavoring | 2.00 |
| Vitamin C | 0.10 |
| Menthol Crystals | 0.03 |
| Malic Acid | 0.60 |
| Sodium Citrate | 5.00 |
| Masker | 1.25 |
| Citric Acid | 0.80 |

TABLE 3
Representative Oral Delivery Composition B

| | Content in weight |
|---|---|
| Component | percent |
| Xylitol | 69.09 |
| Magnesium Stearate | 1.00 |
| Stevioside | 1.00 |
| SNEDDS Composition | 13.53 |
| Sweetmint | 11.00 |
| Sodium Bicarbonate | 1.00 |
| Vitamin C | 0.10 |
| Menthol Crystals | 0.05 |

TABLE 4
Representative Oral Delivery Composition C

| | Content in weight |
|---|---|
| Component | percent |
| Xylitol | 69.04 |
| Magnesium Stearate | 1.00 |
| Stevioside | 1.00 |
| SNEDDS Composition | 13.53 |
| Spearmint | 11.00 |
| Sodium Bicarbonate | 1.00 |
| Vitamin C | 0.10 |
| Menthol Crystals | 0.10 |

TABLE 5
Representative Oral Delivery Composition D

| | Content in weight |
|---|---|
| Component | percent |
| Xylitol | 60.61 |
| Magnesium Stearate | 1.00 |
| Stevioside | 1.00 |
| SNEDDS Composition | 13.53 |
| Citrus | 13.00 |
| Vitamin C | 0.10 |
| Menthol Crystals | 0.03 |
| Citric Acid | 1.25 |
| Sodium Citrate | 5.00 |
| Masker | 1.25 |

The compositions shown in Tables 2-5 are representative. Minor components, processing aids, and rounding account for the difference between the listed components and 100 wt. %, and such differences do not affect the performance of the compositions.

1. An oral delivery product, comprising:
(a) a delivery housing configured for placement in an oral cavity of a user; and
(b) a composition disposed within the delivery housing, the composition comprising:
(i) at least one phytocannabinoid;
(ii) an oil;
(iii) a solid carrier or matrix; and
(iv) a surfactant;
wherein the at least one phytocannabinoid is dissolved or dispersed in the oil;
wherein the oil and the surfactant are associated with the solid carrier or matrix to provide the composition; and
wherein, upon contact with saliva, the composition releases at least a portion of the at least one phytocannabinoid for oral absorption.

2. The oral delivery product of claim 1, wherein the delivery housing comprises a pouch formed from a woven or non-woven fabric.

3. The oral delivery product of claim 1, wherein the delivery housing is saliva-permeable and configured to retain the composition during use.

4. The oral delivery product of claim 1, wherein the solid carrier or matrix comprises a porous inorganic material.

5. The oral delivery product of claim 4, wherein the porous inorganic material comprises an amorphous silica material.

6. The oral delivery product of claim 5, wherein the amorphous silica material comprises a mesoporous silica gel.

7. The oral delivery product of claim 1, wherein the composition is a free-flowing powder.

8. The oral delivery product of claim 1, wherein the oil comprises medium-chain triglyceride (MCT) oil.

9. The oral delivery product of claim 1, wherein the surfactant comprises PEG-40 castor oil, polysorbate 80, or a combination thereof.

10. The oral delivery product of claim 1, wherein the at least one phytocannabinoid comprises tetrahydrocannabinol (THC), cannabidiol (CBD), or a combination thereof.

11. The oral delivery product of claim 1, further comprising at least one sweetener.

12. The oral delivery product of claim 11, wherein the at least one sweetener comprises xylitol, stevioside, or a combination thereof.

13. The oral delivery product of claim 1, further comprising at least one acid, buffer, or pH adjuster.

14. The oral delivery product of claim 13, wherein the at least one acid, buffer, or pH adjuster comprises citric acid, malic acid, sodium citrate, sodium bicarbonate, or a combination thereof.

15. The oral delivery product of claim 1, further comprising at least one masking agent.

16. The oral delivery product of claim 15, wherein the masking agent comprises menthol or menthol crystals.

17. The oral delivery product of claim 1, wherein the oil and the surfactant are present in a mass ratio of from about 1:1 to about 3:1, surfactant to oil.

18. The oral delivery product of claim 1, wherein the composition comprises from about 30 wt. % to about 60 wt. % of the solid carrier or matrix, based on a total mass of components (i)-(iv).

19. A method of preparing the oral delivery product of claim 1, comprising:
(a) combining the at least one phytocannabinoid, the oil, and the surfactant to form a liquid composition;
(b) combining the liquid composition with the solid carrier or matrix to form the composition; and
(c) disposing the composition within the delivery housing.

20. The method of claim 19, wherein step (b) comprises mixing until a dry, free-flowing composition is obtained that is capable of forming a nanoemulsion upon contact with saliva or an aqueous medium.
